(19) United States
(12) Patent Application Publication
(10) Pub. No.: US 2022/0112104 A1
(43) Pub. Date: Apr. 14, 2022

(54) SYSTEM AND METHOD FOR TREATING WASTEWATER

(71) Applicant: Greg Jackson, Chattanooga, TN (US)

(72) Inventor: Greg Jackson, Chattanooga, TN (US)

(21) Appl. No.: 17/500,596

(22) Filed: Oct. 13, 2021

Related U.S. Application Data
(60) Provisional application No. 63/091,197, filed on Oct. 13, 2020.

(51) Int. Cl. C02F 1/461; C02F 1/20; C07C 1/12; F02C 3/20; F02C 3/14; B01D 53/22

(52) U.S. Cl. CPC C02F 1/46104; C02F 1/20; C07C 1/12; C02F 2103/20; F02C 3/14; B01D 53/229; F02C 3/20

(57) ABSTRACT

A system for converting wastewater into natural gas and electricity, said system comprising: (a) a reactor subsystem for receiving said wastewater and converting said wastewater to a first gas output comprising at least one of H2, O2, or CH4 and a second gas output comprising at least H2; (b) a generator subsystem for combusting at least a portion of said first gas output and generating electrical power, said generator subsystem outputting a CO2 stream; and (c) a converter subsystem for converting at least a portion of said CO2 stream and at least a portion of said second gas output to produce a CH4 stream and H20.

### REFERENCE TO RELATED APPLICATION

This application is based on U.S. provisional application No. 63/091,197, filed Oct. 13, 2020, which is hereby incorporated by reference in its entirety, including Appendix A.

### FIELD OF INVENTION

The present application relates, generally, to treating wastewater, and, more specifically, to treating effluent from livestock facilities or refineries to generate electricity and natural gas.

### BACKGROUND

Current processes for treating wastewater, such as effluent from livestock facilities and refinery wastewater are antiquated, and fail to adequately protect the environment. The entire industry, including processors, growers, and handlers, has a sense of urgency to improve wastewater treatment, or face being shut down by (1) significant penalties, (2) public outrage and litigation over health and environmental issues, (3) impending new restrictive legislation regarding disposal, and (4) struggling profitability.

Applicant recognizes the need for new processes that minimize the impact of eliminate biological and refinery wastewater by converting it to a renewable energy source. The present invention fulfils this need among others.

### SUMMARY OF INVENTION

The following presents a simplified summary of the invention in order to provide a basic understanding of some aspects of the invention. This summary is not an extensive overview of the invention. It is not intended to identify key/critical elements of the invention or to delineate the scope of the invention. Its sole purpose is to present some concepts of the invention in a simplified form as a prelude to the more detailed description that is presented later.

In one embodiment, the present invention relates to a system for converting wastewater into natural gas and electricity, the system comprising: (a) a reactor subsystem for receiving the wastewater and converting the wastewater to a first gas output comprising at least one of H2, O2, or CH4 and a second gas output comprising at least H2; (b) a generator subsystem for combusting at least a portion of the first output gas and generating electrical power, the generator subsystem outputting a CO2 stream; and (c) a converter subsystem for converting at least a portion of the CO2 stream and at least a portion of the second gas output to generate a CH4 stream and H20.

In another embodiment, the present invention relates to the use of the system described above.

### BRIEF DESCRIPTION OF FIGURES

FIG. 1 is a schematic of one embodiment of the system of the present invention.

FIG. 2 is a schematic of one embodiment of the system of the present invention.

FIG. 3 is a schematic of an alternative embodiment of the system of the present invention.

FIG. 4 is a schematic of an of one embodiment of a diatomic reactor.

FIG. 5 is a schematic of an of one embodiment of a monoatomic reactor.

### DETAILED DESCRIPTION

In the following paragraphs, the present invention will be described in detail by way of example with reference to the attached drawings. Throughout this description, the preferred embodiment and examples shown should be considered as exemplars, rather than as limitations on the present invention. As used herein, the “present invention” refers to any one of the embodiments of the invention described herein, and any equivalents. Furthermore, reference to various feature(s) of the “present invention” throughout this document does not mean that all claimed embodiments or methods must include the referenced feature(s).

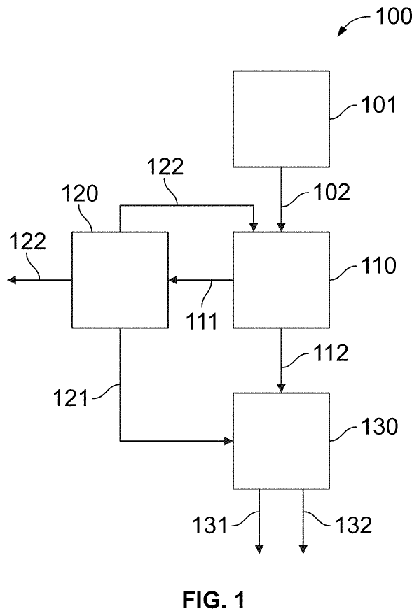

Referring to FIG. 1, one embodiment of a system 100 of the present invention is shown for converting wastewater into natural gas and electricity. In this embodiment, the system 100 comprises a wastewater supply 101 coupled a reactor subsystem 110 for receiving a wastewater stream 102 and converting the wastewater to a first gas output 111 comprising at least one of H2, O2, or CH4 and a second gas output 112 comprising at least H2. The system 100 also comprises a generator subsystem 120 for combusting at least a portion of the first gas output 111 and generating electrical power 122 (which may be used to power the reactor subsystem and/or outputted from the system). The generator subsystem also emits a CO2 stream 121. The system 100 also comprises a converter subsystem 130 for converting at least a portion of the CO2 stream 121 and at least a portion of the second gas output 112 to generate a CH4 stream 131 and an H20 stream 132. These features are considered below in greater detail and in connection with selected embodiments.

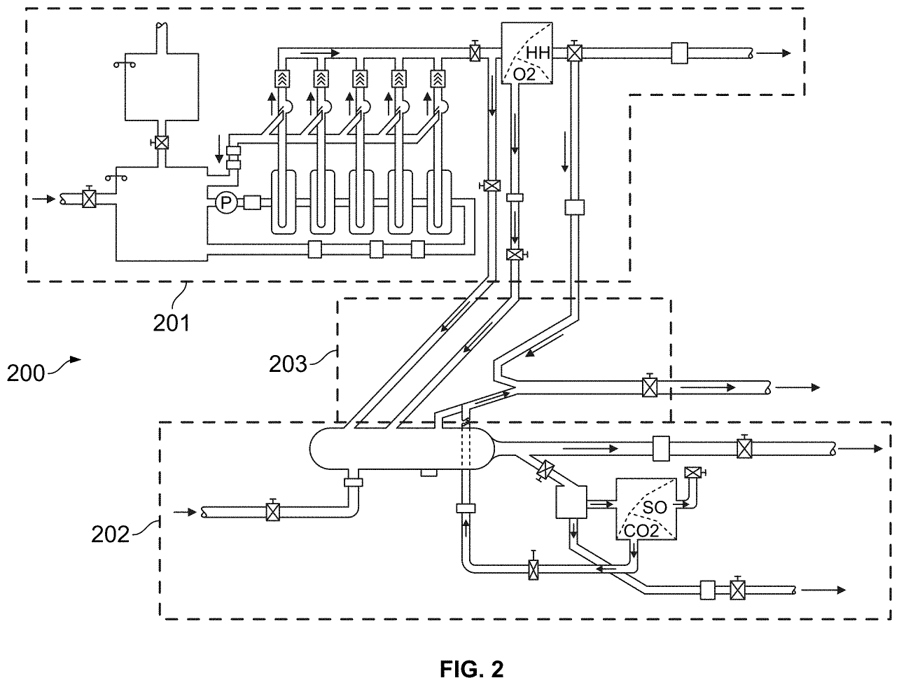

FIG. 2 shows system 200, which is a more detailed embodiment of system 100 of the present invention. For illustrative purposes, the components of system 200 have been partitioned into a reactor subsystem 201, a generator subsystem 202, and a converter subsystem 203. It should be noted that the partitioning of the system is done for illustrative purposes only and should not be construed as limiting the claims. For example, while the partitioning of FIG. 2 may group certain separators and filters as part of the reactor subsystem 201, such components could have just as well been grouped with the generator subsystem 202 or converter subsystem 203.

Reactor Subsystem 201

The reactor subsystem 201 functions to receive the wastewater and convert the wastewater into a first gas output comprising at least a mixture of H2 and O2 (which is referred to herein as an HHO2 mixture), and a second gas output comprising H2.

In the embodiment of FIG. 2, the wastewater 1 is first treated with electrolytes from tank 2 in a holding tank 3 to form a slurry prior to being supplied to the reactor(s). The wastewater may comprise effluent from livestock facilities (including dairy farms, poultry farms, hog farms), plus other facilities that generate wastewater containing chlorine, ammonia, or other alkaline wastewaters, such as refineries. In addition to the wastewater containing chlorine, ammonia, etc., typical biological wastewaters also includes proteins/fats, manure/solids/sludge, and other materials. Such wastewaters are well-known and the detailed chemistry of which will not be considered in detail herein.

The reactor subsystem 201 comprises at least one reactor 9 which functions to convert wastewater into combustible gases. More specifically, in one embodiment, the reactor 9 receives a slurry of wastewater 1 from holding tank 3 and converts the wastewater to an HHO2 mixture and water. In one embodiment, the reactor 9 comprises a diatomic reactor 9. Such reactors are known in the art, and, thus, will not be described in detail herein. Suffice to say that, in one embodiment, a suitable diatomic reactor comprises plastic or nylon encapsulated stainless steel plates, which are spaced apart a specified distance based upon the conductivity of the incoming waste stream—for example, for a highly acidic influent, the plates may be spaced apart 0.300″. The plate size is important in relation to the desired amount of wastewater stream to be processed. For example, a suitable plate configuration for processing 100,000 gallons per 30 days is around 50 plates measuring about 48″×120″×0.1875″. In light of this disclosure, those of skill in the art will be able to size and/configure the plates based on the operating parameters of the system without undue extermination.

During operation, the plates are submerged in wastewater at all times, and an electric current (DC power) is passed through, causing electrolysis and the associated molecular chain reactions which disassociate the constituents of the wastewater to produce at least H2 and O2, and often CH4.

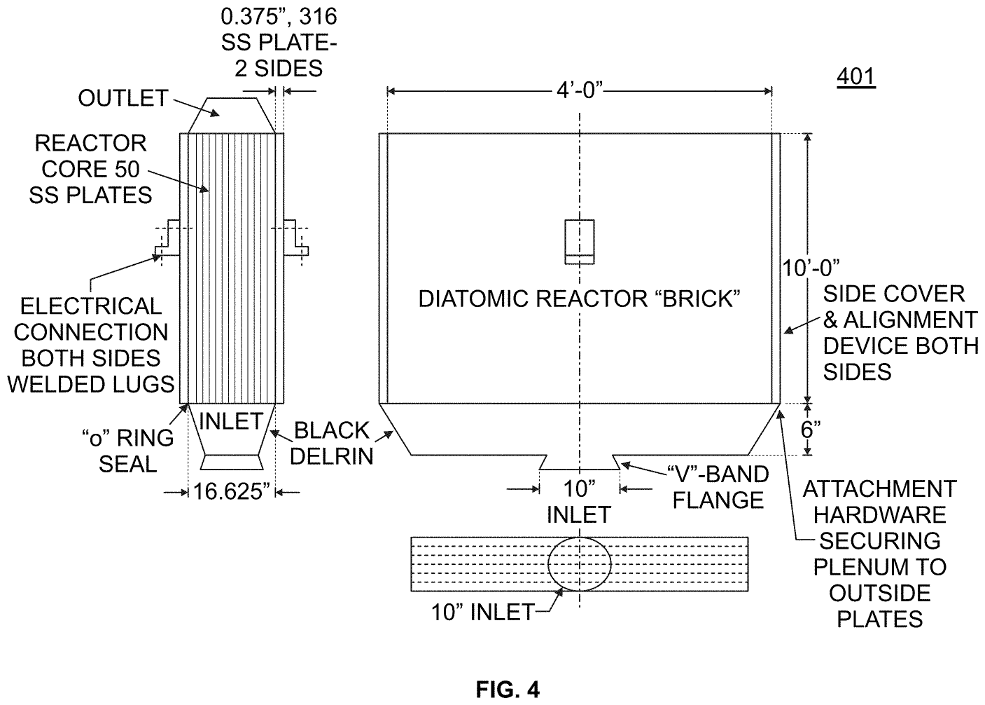

One embodiment of a suitable diatomic reactor is shown in FIG. 4. In this particular embodiment, the diatomic reactor comprises diatomic “bricks.” Each brick comprises a stainless steel reactor plate of suitable thickness (e.g., 0.1875″) encapsulated by two outside plates of suitable thickness allowing two current—carrying lugs, one on each side. The lugs are TIG welded to either side of the stainless steel plate. A bottom plenum accepts the inlet wastewater with flow rate suitable to keep the reactor plates immersed at all times. A top plenum of similar design is used as an outlet for the hydrolyzed water.

In the embodiment of FIG. 2, a plurality of reactors 9 are used. Specifically, in this particular embodiment, five individual reactors 9 are used. Although not necessary, it is generally preferred that the reactors be the same or similar to reduce inventory carrying costs on parts and to simplify maintenance.

In one embodiment, the reactor subsystem 201 comprises a gas/water separator 22. In this particular embodiment, the HHO2 mixture and water exist the reactor 9 and pass through the gas/water separator 22, wherein the water is separated from the HHO2 mixture and returned to the holding tank 3, and the remaining HHO2 mixture is further processes as the first gas output.

In one embodiment, a portion of the first gas output is passed through at least one filter 25 to separate H2 and O2 from the HHO2 mixture to produce an H2 stream and an O2 stream. Although various filter mechanisms may be used, in one embodiment, the filter 25 comprises a membrane system for stripping O2 from the mixture of H2 and O2. Such membrane systems are known and can be commercially sourced from suppliers such as Membrane Systems for Specialty Process Applications (synderfiltration.com) Snyder filtration, Membrane Systems—Novum Structures Novum Structures, and various manufacturers

Although the portion of the first gas output that is filtered may vary based on various factors, including, for example, the composition of the wastewater, the size of the filters, and the required stoichiometry of the converter, in one embodiment, a portion of about 10-100% of the first gas output is filtered, and, in another embodiment, a portion of about 30-80% of the first gas output is filtered, and, in another embodiment, a portion of about 40-60% of the first gas output is filtered.

In one embodiment, the O2 stream from filter 25 is supplied to the at least one generator subsystem 202 as described below, and at least a portion of the H2 stream from filter 25 is fed to the converter subsystem 203 as described below. Depending upon operating conditions and required stoichiometry, the portion of the H2 stream fed to the converter subsystem 203 can vary considerably, for example, from 5 to 100%. In one embodiment, a portion of the H2 stream is outputted from the system for different uses.

Generator Subsystem 202

The generator subsystem functions to generate a CO2 stream and to generate electricity and heat by combusting the HHO2 mixture and O2 from the reactor subsystem. The generator subsystem comprises a gas powered generator 23, which may be, for example, a gas-fired turbine generator or a gas-fired piston generator. Generally, a turbine generator is preferred. As shown in the embodiment of FIG. 2, generator 23 is fed the HHO2 mixture and O2 from the reactor subsystem. In one embodiment, the generator 23 also receives supplemental fuel 29, which may be, for example, natural gas, diesel fuel, kerosene, propane, lng, cng, jet fuel, hydrogen, browns gas, gasoline, toluene, oil, waste oil, yellow oil, cracker bottom oil, C4 industrial waste, crude, or any combination thereof depending upon the application depending upon the application.

The turbine 23 exhausts exhaust gas 26. In this embodiment, a portion of the exhaust gas 26 is diverted to isolate a CO2 stream. More specifically, in this embodiment, a portion of the exhaust gas 26 is fed into a chiller 19 which cools the portion of exhaust gas to precipitate/separate water from the exhaust gas. This is important since the membrane stripping of CO2 (as described below) is much more efficient when receiving dry gasses. The separated water, in one embodiment, may be purified (e.g., to a PH of 7) so it can be reused or pumped into a drainage system.

After the water is separated from a portion of the exhaust gas, the dry exhaust gas is filtered in filter 25 to separate CO2 from the other exhaust gases, which may include, for example, SOx and unburnt fuel, to form a CO2 stream and a waste exhaust stream. As with the filter 25 used to separate H2 from O2 described above, in one embodiment, filter 25 is a membrane filter, which is known to those of skill in the art in light of this disclosure. The waste exhaust, e.g., SOx and unburnt fuel, are exhausted from the system, while the CO2 stream is fed to the converter subsystem 203 as described below. Alternatively, rather than using a membrane filter, in one embodiment, the CO2 stream is cryogenically cooled to form dry ice, thereby separating the CO2 from impurities in the exhaust stream(s) of the generator. The dry ice then is allowed to vaporize and react with H2 from the second gas output stream of the reactor.

In addition to exhausting an exhaust stream, the generator 203 also generates electricity, which is used, in one embodiment, to support the power requirements of the system, and, more particularly, the reactor subsystem. Alternatively, the electricity generated by the generator 203 can be outputted for the system for use on the grid or private use. In one embodiment, the electricity generated by the generator is direct current (DC) power. Such an embodiment may be preferred is used to power the diatomic reactor because electrolysis requires DC power. If, however, there is desire to use the power for other purposes, it may be preferable to generate alternating current (AC) power, given the preference for AC power for electrical power. In such an embodiment, it may be preferable to incorporate a rectifier or similar circuitry for converting a portion of the AC power to DC power for use in the diatomic reactor. In yet another embodiment, the generator may be configured as a turbine that turns a DC generator and an AC generator to output both DC and AC power. Still other embodiments will be obvious to those of skill the art in light of this disclosure.

The generator 23 also produces heat, which, in one embodiment, the converter subsystem 203 uses to heat the CO2 stream as described below.

Converter Subsystem 203

The converter subsystem 203 functions to react CO2 with H2 to form CH4 and water. In one embodiment, the reaction is based on the well-known Sabatier process. Suffice to say that, in the Sabatier process, H2 and CO2 are introduced together at certain temperatures and pressures, at certain mixture ratios, to combine to form CH4 and water. The mixing of the CO2 and H2 is controlled via orifices, reducing cones, diverging nozzles, valving, and pumping systems to achieve the correct stoichiometry. In one embodiment, the CO2 stream from the generator subsystem is combined with the H2 stream from the reactor subsystem using an orificed mixing nozzle 24. The mixing nozzle configuration may change depending upon the wastewater or feedstock initially introduced among other factors. Other embodiments will be known to those of skill in the art in light of this disclosure.

In one embodiment, the CO2 stream from the generator subsystem is heated by the heat generated from the turbine 23 in a heat exchanger 16 before being reacted with the H2 stream. In one embodiment, this CO2 stream is heated to a temperature of approximately seven hundred and eighty (780) degrees F. At this temperature, the CO2 will combine in the mixing nozzle with hydrogen thus providing CH4. The CH4 may be outputted from the system or to be used as supplemental fuel to be combusted by the generator as described above. Alternatively, electrical power from the generator may be used to heat and pressurize the CO2. Still other power sources for facilitating the conversion will be obvious to those of skill he art in light of this disclosure.

Although certain components of the reactor, generator, and converter subsystems are described above, it should be understood that system 200 comprises other components to optimize the system and method of the present invention. For example, throughout the system, various flow control valves 5 are used to control the metering of certain streams. For example, such valves are used to control the portion of the first gas stream that is filtered to produce the second gas stream; the portion of the exhaust stream that is converted to the CO2 stream; the amount of HHO2 mixture and O2 fed to the generator subsystem; and the amount of H2 and CO2 fed to the converter subsystem. These flow control valves 5 allow for essentially infinite variability of flowrates. Additionally, throughout system 200 are the following: a liquid level switch 4, pump 6, backflow preventer 7, pH sensor 8, AMP meter-voltmeter-pH sensor 10, VOC sensor, O2 sensor, 3-way valve 14, CH4 sensor 15, temperature sensor 17, engine data 18, HH sensor 20, differential pressure sensor 21, caloric (natural gas) sensor 26, CO2 sensor 27, return water flange 27, and CO2 into heat exchanger 28.

### Alternative Embodiment

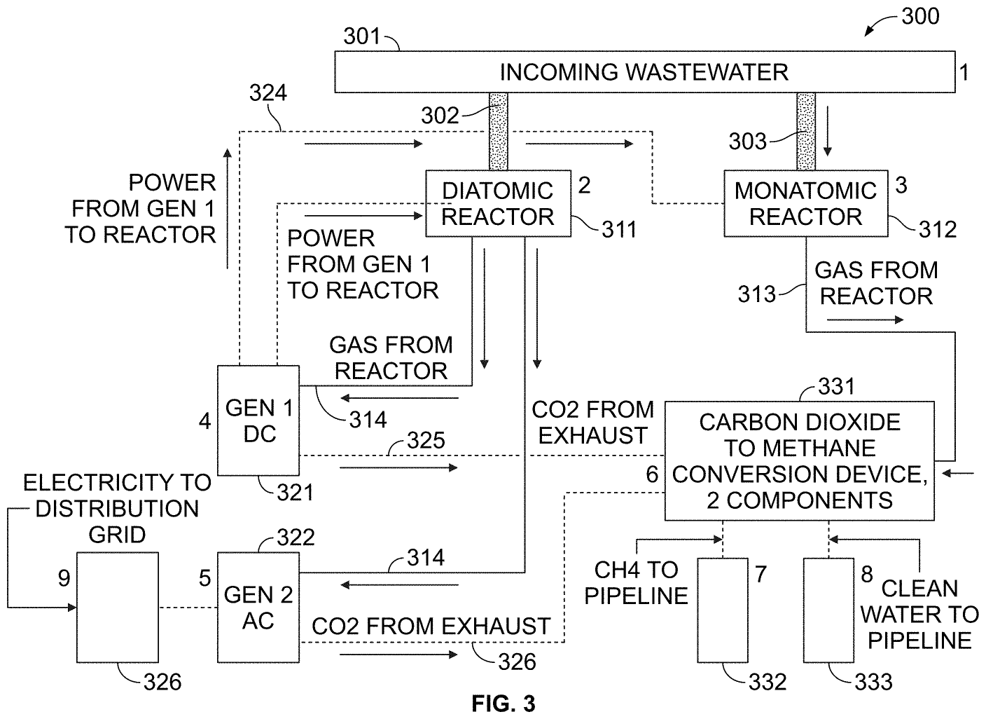

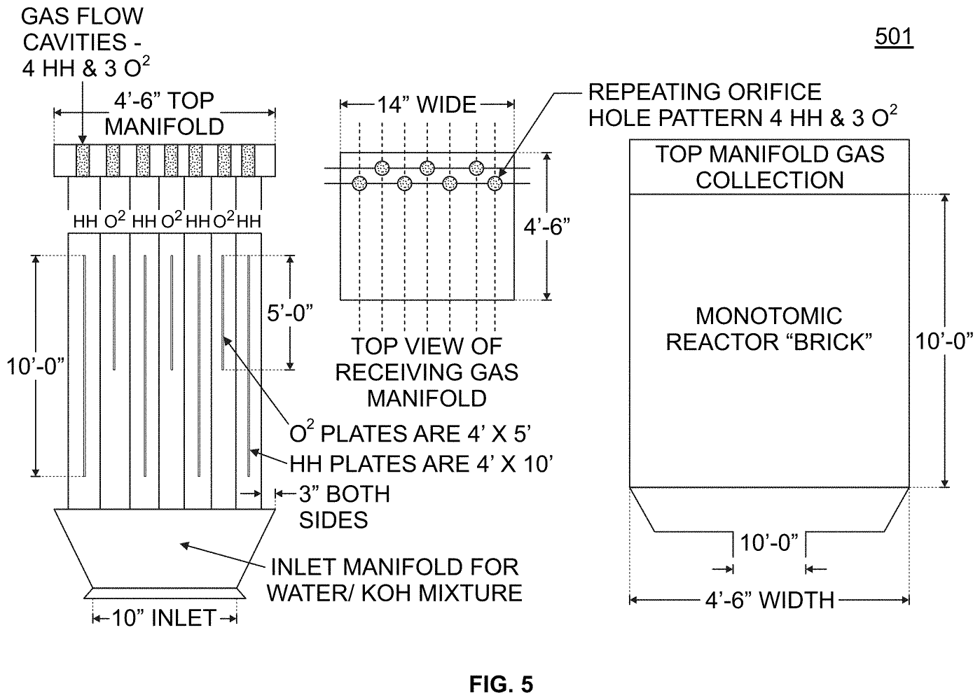

FIG. 3 shows system 300, which is an alternative detailed embodiment of system 100 of the present invention. In this embodiment, the reactor subsystem comprises at least two types of reactors, a first reactor for outputting the first gas output and a second reactor for outputting the second gas output. For example, referring to FIG. 3, a more particular embodiment of a system 300 of the present invention is shown. In this embodiment, the first reactor is a diatomic reactor 311 and the second reactor is a monoatomic reactor 312. The configuration of the monoatomic and diatomic reactors may vary. For example, in one embodiment, the monoatomic and diatomic reactors comprise monoatomic and diatomic “bricks.” The bricks comprise stainless steel reactor plates of suitable thickness (e.g., 0.1875″) encapsulated by two outside plates of suitable thickness allowing two current—carrying lugs, one on each side. The lugs are TIG welded to either side of the stainless steel plate. A bottom plenum accepts the inlet wastewater with flow rate suitable to keep the reactor plates immersed at all times. A top plenum of similar design is used as an outlet for the hydrolyzed water. Particular embodiments the diatomic reactor 401 and the monoatomic reactor 501 are shown in FIGS. 4 and 5, respectively

In one embodiment, the reactor subsystem further comprising at least one separator for separating the wastewater into at least two streams, a first stream 302 comprising any solids from the wastewater, and a second stream 303 containing no solids, wherein the first stream feeds the diatomic reactor 311 and the second stream feeds the monoatomic reactor 312. The configuration of such a separator may vary. For example, in one embodiment, the separator comprises a settling tank in which the top effluent feeds the monoatomic reactor and the bottom effluent feeds the diatomic reactor. Still other embodiments are possible. For example, a centrifuge may be used to separate solids from the wastewater.

In one embodiment, a portion 324 of the electrical power is used to power the monoatomic reactor 312, and another portion 323 of the electrical power is used to power the diatomic reactor 311 as shown in FIG. 3. In one embodiment, only a portion of the electrical power is used by the at least one reactor, thus leaving surplus electrical power 326 which can be sold back to the grid.

In one embodiment, the generator subsystem comprises at least one generator. In one embodiment, the at least one generator comprises at least two types of generators, a first type for providing power to the at least one reactor, and a second type for outputting power from the system. For example, referring to FIG. 3, the first type is a DC generator 321, and the second type is an AC generator 322. In certain embodiments, it may be beneficial to generate DC power directly because DC power is needed for the electrolysis in the at least one reactor. Although it may be preferred to generate DC power directly, it should be understood that other embodiments are possible. For example, a rectifier may be used to convert a portion of AC power from the AC generator to generate DC power.

In one embodiment, the converter subsystem uses the Sabatier process to convert CO2 into methane and water. Although this may be accomplished in different ways, in one embodiment, the CO2 stream is cryogenically cooled to form dry ice, thereby separating the CO2 from impurities in the exhaust stream(s) of the generator. The dry ice then is allowed to vaporize and react with H2 from the second gas output stream of the reactor. Specifically, referring to the embodiment of FIG. 3, the converter 331 receives exhaust gas streams 325, 326 from the DC generator 321 and the AC generator 322, respectively, as shown. The converter 331 also receives H2 from the second gas output 313 from the monoatomic reactor 312. Using the Sabatier process, the converter 331 produces methane or natural gas 332 and water 333. In one embodiment, the converter also outputs an O2 stream. In one particular embodiment, at least a portion of the O2 stream is used for combustion in the at least one generator.

In one particular embodiment, the converter subsystem is powered by heat being emitted from the at least one generator. For example, heat from the turbocharger of one or more generators can be used to facilitate the Sabatier process. Alternatively, electrical power from at least one generator may be used. Still other power sources will be obvious to those of skill he art in light of this disclosure.

Having thus described a few particular embodiments of the invention, various alterations, modifications, and improvements will readily occur to those skilled in the art. Such alterations, modifications, and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

1. A system for converting wastewater into natural gas and electricity, said system comprising:
a reactor subsystem for receiving said wastewater and converting said wastewater to a first gas output comprising at least one of H2, O2, or CH4 and a second gas output comprising at least H2;
a generator subsystem for combusting at least a portion of said first gas output and generating electrical power, said generator subsystem outputting a CO2 stream; and
a converter subsystem for converting at least a portion of said CO2 stream and at least a portion of said second gas output to produce a CH4 stream and H20.

2. The system of claim 1, wherein said reactor subsystem comprises at least one reactor for outputting said first gas output, said first gas output comprising at least a HHO2 mixture, and at least one filter for filtering a portion of said first gas output to output said second gas stream.

3. The system of claim 2, wherein said at least one reactor comprises at least one diatomic reactor.

4. The system of claim 3, wherein said at least one diatomic reactor comprises a plurality of diatomic reactors.

5. The system of claim 2, wherein said reactor subsystem comprising at least one gas/water separator for separating water from said HHO2 mixture.

6. The system of claim 5, wherein at least one filter comprises a membrane system for stripping O2 from said HHO2 mixture to form an O2 stream and an H2 stream.

7. The system of claim 6, wherein at least a portion of said O2 stream is supplied to said generator subsystem for combustion.

8. The system of claim 1, wherein at least a portion of said electrical power is used by said reactor subsystem.

9. The system of claim 1, wherein said generator subsystem comprises at least one gas turbine generator.

10. The system of claim 1, wherein said generator subsystem comprises an AC generator for outputting AC power, and a rectifier for converting a portion of said AC power to DC power for powering said reactor subsystem.

11. The system of claim 1, wherein said generator subsystem comprises a DC generator for outputting DC power.

12. (canceled)

13. The system of claim 1, wherein said generator subsystem comprises at least one generator which exhausts an exhaust stream, and wherein said generator subsystem comprises a filter to filter a portion of said exhaust stream to produce said CO2 stream.

14. The system of claim 13, wherein said filter is a membrane separator to separate CO2 from said at least a portion of said exhaust stream.

15. The system of claim 1, wherein said converter subsystem produces said CH4 stream and H2O using the Sabatier reaction.

16. The system of claim 1, wherein heat from said generator subsystem is used by said converter subsystem.

17. A method of using the system of claim 1.

18. The method of claim 17, wherein said wastewater comprises organic waste or refinery wastewater.

19. The method of claim 17, wherein said organic waste comprises effluent from livestock.

20. (canceled)

21. The method of claim 17, wherein said wastewater is treated with electrolytes in a holding tank to form a slurry prior to being supplied to said at least one reactor.

22. The method of claim 17, wherein said portion of said first gas output is about 10-80% of said first gas output.

23. (canceled)

24. (canceled)

25. (canceled)
